(19) United States
(12) Patent Application Publication
(10) Pub. No.: US 2008/0050781 A1
(43) Pub. Date: Feb. 28, 2008

(54) Systems and Methods for Cooling in a Thermal Cycler

(75) Inventors: Mark F. Oldham, Los Gatos, CA (US); Eric G. Henderson, Fremont, CA (US); Vinod Mirchandani, San Ramon, CA (US); Eric S. Nordman, Palo Alto, CA (US); Marc Haberstroh, San Jose, CA (US); Johannes P. Sluis, Redwood City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/767,327

(22) Filed: Jun. 22, 2007

Related U.S. Application Data
(60) Provisional application No. 60/816,192, filed on Jun. 23, 2006.
(60) Provisional application No. 60/816,133, filed on Jun. 23, 2006.

(51) Int. Cl. C12P 19/34; B01J 19/00; C12M 1/38

(52) U.S. Cl. 435/91.2; 422/105; 435/286.1

(57) ABSTRACT

A device for performing polymerase chain reactions using cooling members not used in conventional thermal cyclers. A device for performing polymerase chain reactions may include a sample holder configured to receive a nucleic acid sample, a heating system configured to raise the temperature of the sample, a cooling system configured to lower the temperature of the sample, and a controller configured to operably control the heating system and the cooling system to cycle the device through a desired time-temperature profile. The cooling system may comprise at least one cooling member selected from a synthetic jet ejector array, a vibration-induced droplet atomization system, a vibrating diaphragm system, a piezo fan, a Cold Gun, a microchannel cooling loop, and a Cool Chip.

### CROSS REFERENCE TO RELATED APPLICATIONS

This application claims a priority benefit under 35 U.S.C. §119(e) from U.S. Patent Application No. 60/816,192 filed Jun. 23, 2006 and Application No. 60/816,133 filed Jun. 23, 2006, all of which are incorporated herein by reference.

### FIELD

This disclosure pertains generally to instruments for performing polymerase chain reactions (PCR). More particularly, this disclosure is directed to systems and methods for cooling in a thermal cycler configured to perform polymerase chain reactions substantially simultaneously on a plurality of samples.

### INTRODUCTION

To amplify DNA (Deoxyribose Nucleic Acid) using the PCR process, a specially constituted liquid reaction mixture is cycled through a PCR protocol that includes several different temperature incubation periods. The reaction mixture is comprised of various components such as the DNA to be amplified and at least two primers selected in a predetermined way so as to be sufficiently complementary to the sample DNA as to be able to create extension products of the DNA to be amplified. The reaction mixture includes various enzymes and/or other reagents, as well as several deoxyribonucleoside triphosphates such as dATP, dCTP, dGTP and dTTP. Generally, the primers are oligonucleotides which are capable of acting as a point of initiation of synthesis when placed under conditions in which synthesis of a primer extension product which is complimentary to a nucleic acid strand is induced, i.e., in the presence of nucleotides and inducing agents such as thermostable DNA polymerase at a suitable temperature and pH.

A significant aspect to PCR is the concept of thermal cycling; that is, alternating steps of melting DNA, annealing short primers to the resulting single strands, and extending those primers to make new copies of double stranded DNA. In thermal cycling, the PCR reaction mixture is repeatedly cycled from high temperatures of about 90° C. for melting the DNA, to lower temperatures of approximately 40° C. to 70° C. for primer annealing and extension. The details of the polymerase chain reaction, the temperature cycling and reaction conditions necessary for PCR as well as the various reagents and enzymes necessary to perform the reaction are described in U.S. Pat. Nos. 4,683,202, 4,683,195, and 4,889,818, and in EPO Publication 258,017, the entire disclosures of which are hereby incorporated by reference herein.

The purpose of a polymerase chain reaction is to manufacture a large volume of DNA which is identical to an initially supplied small volume of “seed” DNA. The reaction involves copying the strands of the DNA and then using the copies to generate other copies in subsequent cycles. Under ideal conditions, each cycle will double the amount of DNA present thereby resulting in a geometric progression in the volume of copies of the “target” or “seed” DNA strands present in the reaction mixture.

A typical PCR temperature cycle requires that the reaction mixture be held accurately at each incubation temperature for a prescribed time and that the identical cycle or a similar cycle be repeated many times. A typical PCR program starts at a sample temperature of about 94° C. held for 30 seconds to denature the reaction mixture. Then, the temperature of the reaction mixture is lowered to about 37° C. and held for one minute to permit primer hybridization. Next, the temperature of the reaction mixture is raised to a temperature in the range from about 50° C. to about 72° C., where it is held for two minutes to promote the synthesis of extension products. This completes one cycle. The next PCR cycle then starts by raising the temperature of the reaction mixture to about 94° C. again for strand separation of the extension products formed in the previous cycle (denaturation). Typically, the cycle is repeated 25 to 30 times.

Generally, it is desirable to change the sample temperature to the next temperature in the cycle as rapidly as possible for several reasons. First, the chemical reaction has an optimum temperature for each of its stages. Thus, less time spent at non-optimum temperatures may achieve a better chemical result. Another reason is that a minimum time for holding the reaction mixture at each incubation temperature is required after each said incubation temperature is reached. These minimum incubation times establish the “floor” or minimum time it takes to complete a cycle. Any time transitioning between sample incubation temperatures is time added to this minimum cycle time. Since the number of cycles is fairly large, this additional time undesirably lengthens the total time needed to complete the amplification.

In some conventional automated PCR instruments, to perform the PCR process, the temperature of a metal block which holds containers, holders, or the like containing samples, is controlled according to prescribed temperatures and times specified by the user in a PCR protocol file. A computer and associated electronics control the temperature of the metal block in accordance with the user supplied data in the PCR protocol file defining the times, temperatures and number of cycles, etc. As the metal block changes temperature, the samples held in the various sample containers or holders may follow with similar changes in temperature. However, in these conventional instruments not all samples experience the same temperature cycle. In these conventional PCR instruments, errors in sample temperature may be generated by nonuniformity of temperature from place to place within the metal sample block, i.e., temperature variability exists within the metal of the block thereby undesirably causing some samples to have different temperatures than other samples at particular times in the cycle. Further, there may be delays in transferring heat from the block to the sample, but the delays may not be the same for all samples.

In other conventional automated PCR systems, sample holders, for example, capillaries, may be heated and/or cooled without the use of a metal block. For example, in such systems, air or other fluid may be circulated directly around the holders. The temperature of the samples in such systems also may be relatively difficult to control, e.g., such that all of the samples reach the same temperature and/or change temperatures substantially simultaneously. In other words, in such systems, undesirable temperature variations among the samples may occur. Further, it may be difficult to change the temperature of the samples in an efficient manner using direct cooling and/or heating via circulating fluid.

To perform the PCR process successfully and efficiently, and to enable so called “quantitative” PCR, it is desirable to minimize such time delays and temperature errors (e.g., undesirable temperature variations) that may occur in conventional systems.

The problems of minimizing time delays for heat transfer to and from the samples and minimizing temperature errors due to undesirable temperature variability (nonuniformity) may become particularly acute when the size of the region containing samples becomes large. It is a desirable attribute for a PCR instrument to be configured to accommodate sample holders (e.g., tubes, wells, containers, recesses, capillaries, sample locations, etc., for example, of microtiter plates, microcards, individual capillary tubes.) that comply with industry standard formats in both number and arrangement (e.g., 48-, 96-, 384-, 768-, 1536-, 6144- etc. holder format).

One widely used means for handling, processing and analyzing large numbers of small (e.g., microvolume) samples in the biochemistry and biotechnology fields includes the microtiter plate. In an exemplary arrangement, a microtiter plate is a tray which is 35/8 inches wide and 5 inches long and contains 96 identical sample wells in an 8 well by 12 well rectangular array on 9 millimeter centers. Although microtiter plates are available in a wide variety of materials, shapes, volumes, and numbers of the sample wells, which are optimized for many different uses, microtiter plates typically have the same overall outside dimensions. A wide variety of equipment is available for automating the handling, processing and analyzing of samples in this standard microtiter plate format. Although 96-well plate formats are commonly used, microtiter plates in other formats also may be used, including, for example, 48-, 384, 768-, 1536-, 6144- etc. well formats.

Furthermore, there are numerous other types of sample holders that may be used in lieu of microtiter plates. By way of example only, samples may be held in a plurality of capillaries, capped disposable tubes, and in various flat microcards where plural samples are collected at predetermined locations on the surface of the microcard.

It is therefore a desirable characteristic for a PCR instrument to be able to perform the PCR reaction on numerous samples simultaneously, wherein the samples are arranged and held in a format, such as, for example, any of the various formats discussed above and known to those having skill in the art.

When using a metal block to conduct heat with the samples, the size of such a block which is necessary to heat and cool, for example, at least 96 samples in an 8×12 well array on 9 millimeter centers is fairly large. This large area block creates multiple challenging engineering problems for the design of a PCR instrument that is capable of heating and cooling such a block very rapidly in a temperature range generally from 0° C. to 100° C. and with very little tolerance for temperature variations between samples. These problems arise from several sources. First, the large thermal mass of the block makes it difficult to move the block temperature up and down in the operating range with great rapidity. Second, in some conventional instruments, the need to attach the block to various external devices such as manifolds for supply and withdrawal of cooling fluid, block support attachment points, and associated other peripheral equipment creates the potential for temperature variations to exist across the block which exceed tolerable limits.

There are also numerous other conflicts between the requirements in the design of a thermal cycling system for automated performance of the PCR reaction or other reactions requiring rapid, accurate temperature cycling of a large number of samples. For example, to change the temperature of a metal block and/or the samples rapidly, a large amount of heat must be added to, or removed from the block and/or the samples in a short period of time. In some conventional instruments, heat can be added from electrical resistance heaters, while in others, heat can be added by flowing a heated fluid in contact with the block. Similarly, in some conventional instruments, heat can be removed by flowing a chilled fluid in contact with the block and/or the sample holders, while in others, heat can be removed by a heat sink and fan combination. However, it may be difficult to add or remove large amounts of heat rapidly and efficiently by these means without causing large differences in temperature from place to place in the block and/or the sample holders thereby forming temperature variability which can result in nonuniformity of temperature among the samples.

Even after the process of addition or removal of heat is terminated, temperature variability can persist for a time roughly proportional to the square of the distance that the heat stored in various points in the block must travel to cooler regions to eliminate the temperature variance. Thus, as a metal block is made larger to accommodate more samples, the time it takes for temperature variability existing in the block to decay after a temperature change causes temperature variance which extends across the largest dimensions of the block can become markedly longer. This makes it increasingly difficult to cycle the temperature of the sample block rapidly while maintaining accurate temperature uniformity among all the samples.

Because of the time required for temperature variations to dissipate, an important need has arisen in the design of a high performance PCR instrument to prevent the creation of undesired temperature variablity that may extend over large distances. Thus, it may be desirable to provide a thermal cycler for performing PCR, wherein the sample block can be cooled in a rapid, efficient, and uniform manner. It also may be desirable to provide a thermal cycler for performing PCR wherein the sample holders can be directly cooled and/or heated in an efficient and rapid manner, for example, without the use of a metal block. It may be desirable to provide a thermal cycler that is capable of achieving sub-ambient temperatures.

On the other hand, there may be a need in some applications of a thermal cycler to create desired temperature gradients among the samples, e.g., at certain locations of the sample holders or sample block. Thus, it may be desirable to provide a thermal cycler with a cooling system capable of creating desired temperature gradients (e.g, controlled temperature gradients).

### SUMMARY

The present invention may satisfy one or more of the above-mentioned desirable features. Other features and/or advantages may become apparent from the description which follows.

According to various exemplary aspects of the disclosure, a device for performing polymerase chain reactions in a nucleic acid sample may comprise a sample holder configured to receive a nucleic acid sample, a heating system configured to raise the temperature of the sample, a cooling system configured to lower the temperature of the sample, and a controller configured to operably control the heating system and the cooling system to cycle the device through a desired time-temperature profile. The cooling system may comprise at least one cooling member.

According to some exemplary aspects of the disclosure, a device for performing polymerase chain reactions in a nucleic acid sample may comprise a sample holder configured to receive a nucleic acid sample, a heating system configured to raise the temperature of the sample, a cooling system configured to lower the temperature of the sample, and a controller configured to operably control the heating system and the cooling system to cycle the device through a desired time-temperature profile. The cooling system may comprise at least one cooling member selected from a synthetic jet ejector array, a vibration-induced droplet atomization system, a vibrating diaphragm system, a piezo fan, a Cold Gun, a microchannel cooling loop, and a Cool Chip.

In accordance with various exemplary aspects of the disclosure, a device for performing polymerase chain reactions in a nucleic acid sample may comprise means for holding a nucleic acid sample, means for heating the sample, means for cooling the sample, and means for controlling the means for heating and the means for cooling to cycle the device through a desired time-temperature profile. The means for cooling the sample may comprise a heat sink and a means for cooling the heat sink, wherein the means for cooling the heat sink comprises a cooling member.

In accordance with various exemplary aspects of the disclosure, a device for performing biological sample processing may comprise: a sample holder configured to receive a biological sample; a heating system configured to raise the temperature of the sample; a cooling system configured to lower the temperature of the sample, wherein the cooling system comprises at least one cooling member; and a controller configured to operably control the heating system and the cooling system to cycle the device through a desired time-temperature profile.

In accordance with various exemplary aspects of the disclosure, a device for performing biological sample processing may comprise: a sample holder configured to receive a biological sample; a heating system configured to raise the temperature of the sample; a cooling system configured to lower the temperature of the sample, wherein the cooling system comprises at least one cooling member selected from a synthetic jet ejector array, a vibration-induced droplet atomization system, a vibrating diaphragm system, a piezo fan, a Cold Gun, a microchannel cooling loop, and a Cool Chip; and a controller configured to operably control the heating system and the cooling system to cycle the device through a desired time-temperature profile.

In accordance with various exemplary aspects of the disclosure, a device for performing biological sample processing may comprise: means for holding a biological sample; means for heating the sample; means for cooling the sample, wherein the means for cooling the sample comprises a heat sink and a means for cooling the heat sink, wherein the means for cooling the heat sink comprises a cooling member; and means for controlling the means for heating and the means for cooling to cycle the device through a desired time-temperature profile.

In accordance with various exemplary aspects of the disclosure, a method for performing biological sample processing may comprise: supplying an enclosure with a biological sample for processing within the enclosure; modulating a temperature of the biological sample to cycle a temperature of the biological sample,wherein modulating the temperature of the biological sample comprises respectively directing a cooling fluid via a plurality of separate flow passages to a plurality of locations of a heat sink in thermal communication with the enclosure, wherein the plurality of locations are independently cooled via the cooling fluid respectively directed to each of the plurality of locations.

In the following description, certain aspects and embodiments will become evident. It should be understood that the invention, in its broadest sense, could be practiced without having one or more features of these aspects and embodiments. It should be understood that these aspects and embodiments are merely exemplary and explanatory and are not restrictive of the invention.

### BRIEF DESCRIPTION OF THE DRAWINGS

FIG. 1A is a block diagram of a thermal cycler in accordance with an exemplary embodiment;

FIG. 1B is a block diagram of a thermal cycler in accordance with another exemplary embodiment;

FIG. 2 is a cross-sectional view of a portion of an exemplary embodiment of a sample block of a thermal cycler;

FIG. 3 is a side, elevational view of an exemplary embodiment of a thermal electric device;

FIG. 4 is a cut-away, partial, isometric view of an exemplary embodiment of a heat sink;

FIG. 5 is a block diagram of an exemplary embodiment of a cooling system of a thermal cycler in accordance with aspects of the disclosure;

FIG. 6 is a block diagram of an exemplary embodiment of a cooling system of a thermal cycler in accordance with aspects of the disclosure;

FIG. 7 is a block diagram of an exemplary embodiment of a cooling system of a thermal cycler in accordance with aspects of the disclosure;

FIG. 8 is a block diagram of an exemplary heat sink, carbon block, and sample block in accordance with aspects of the disclosure;

FIGS. 9a-9b is a view of exemplary embodiments of the carbon block taken along line IX-IX of FIG. 8; and

FIG. 10 is a block diagram of yet another exemplary embodiment of a cooling system of a thermal cycler in accordance with aspects of the disclosure.

### DETAILED DESCRIPTION OF VARIOUS EMBODIMENTS

Reference will now be made to various embodiments, examples of which are illustrated in the accompanying drawings. However, these various exemplary embodiments are not intended to limit the disclosure. On the contrary, the disclosure is intended to cover alternatives, modifications, and equivalents.

With respect to containers, holders, chambers, wells, recesses, tubes, capillaries and/or locations used in conjunction with plates, trays, cards, and/or alone, as used herein, such structures may be “micro” structures, which refers to the structures being configured to hold a small (micro) volume of fluid; e.g., no greater than about 250 μl to about 300 μl. In various embodiments, such structures are configured to hold no more than 100 μl, no more than 75 μl, no more than 50 μl, no more than 25 μl, or no more than 1 μl. In some embodiments, such structures can be configured to hold, for example, about 30 μl.

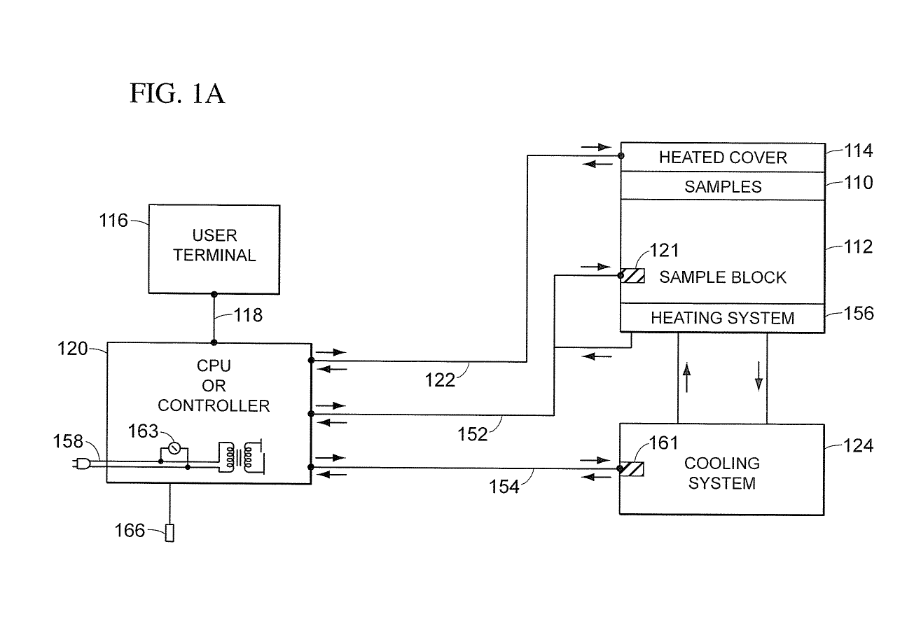

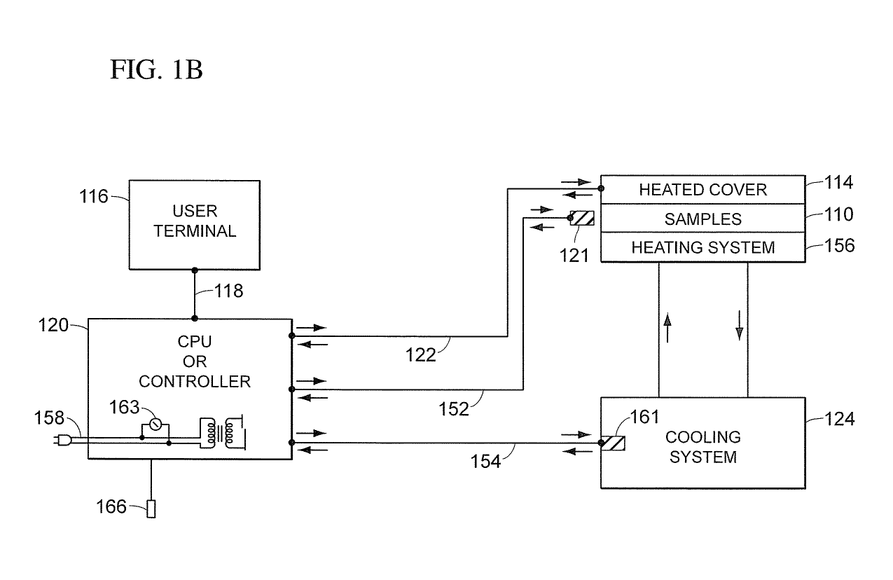

Referring to FIGS. 1A and 1B, a block diagram of the major system components of exemplary embodiments of a thermal cycler for performing PCR according to the exemplary aspects of the disclosure is shown. With reference to FIG. 1A, sample mixtures, including the DNA to be amplified, are placed in the temperature-programmed sample block 112 and are covered by heated cover 114. The sample block may be a metal block constructed, for example, from silver. With reference to FIG. 1B, another exemplary embodiment of a thermal cycler for performing PCR is illustrated. This embodiment does not include a sample block. Rather, the samples are directly heated and/or cooled.

With either embodiment, a user may supply data defining time and temperature parameters (e.g., time-temperature profiles) of the desired PCR protocol via a terminal 116 including a keyboard and display. The keyboard and display are coupled via a data bus 118 to a controller 120 (sometimes referred to as a central processing unit or CPU). The controller 120 can include memory that stores a desired control program, data defining a desired PCR protocol, and certain calibration constants. Based on the control program, the controller 120 controls temperature cycling of the sample block 112 and/or holders containing the samples 110 and implements a user interface that provides certain displays to the user and receives data entered by the user via the keyboard of the terminal 116. It should be appreciated that the controller 120 and associated peripheral electronics to control the various heaters and other electro-mechanical systems of the thermal cycler and read various sensors can include any general purpose computer such as, for example, a suitably programmed personal computer or microcomputer.

Samples 110 can be held in a sample holder (e.g., in microcards, microplates, capillaries, etc.) configured to be seated in the sample block 112 and thermally isolated from the ambient air by the heated cover 114, which contacts a plastic disposable tray to form a heated, enclosed box in which the sample holders reside. The sample holders may include, for example, recesses and/or wells in a microtiter plate, capillaries, locations for holding samples on a microcard, and/or other conventional sample holders used for PCR processes. The heated cover serves, among other things, to reduce undesired heat transfer to and from the sample mixture by evaporation, condensation, and refluxing inside the sample tubes. It also may reduce the chance of cross contamination by maintaining the insides of the caps of capillary tubes dry thereby preventing aerosol formation when the tubes are uncapped. The heated cover may be in contact with the sample tube caps and/or other sealing mechanism over the sample holders so as to keep them heated to a temperature of approximately 104° C. or above the condensation points of the various components of the reaction mixture.

The controller 120 can include appropriate electronics to sense the temperature of the heated cover 114 and control electric resistance heaters therein to maintain the cover 114 at a predetermined temperature. Sensing of the temperature of the heated cover 114 and control of the resistance heaters therein is accomplished via a temperature sensor (not shown) and a data bus 122.

A cooling system 124, examples of which are discussed in more detail below, can provide precise temperature control of the samples 110. According to some aspects, the cooling system 124 can be operated to achieve fast, efficient, and/or uniform temperature control of the samples 110. According to some aspects, the cooling system 124 can be operated to quickly and/or efficiently achieve a desired temperature gradient between various samples.

According to various aspects, the apparatus of FIGS. 1A and 1B can be enclosed within a housing (not shown). Any heat being expelled to the ambient air can be kept within the housing to aid in evaporation of any condensation that may occur. This condensation can cause corrosion of metals used in the construction of the unit or the electronic circuitry and should be removed. Expelling the heat inside the enclosure helps evaporate any condensation to prevent corrosion.

As noted above, the PCR protocol may involve incubations at at least two different temperatures and often three different temperatures. These temperatures are substantially different, and, therefore means must be provided to move the temperature of the reaction mixture of all the samples rapidly from one temperature to another. The cooling system 124 is configured to reduce the temperature of the samples 110 from the high temperature denaturation incubation to the lower temperature hybridization and extension incubation temperatures. For example, the cooling system 124 may lower the temperature of the sample block 112 (FIG. 1A) or may act to directly lower the temperature of holders containing the samples 110 (FIG. 1B),

It should be appreciated that a ramp cooling system, in some exemplary embodiments, may also be used to maintain the sample temperature at or near the target incubation temperature. However, in some embodiments, small temperature changes in the downward direction to maintain target incubation temperature are implemented by a bias cooling system (e.g., a Peltier thermoelectric device), as is known to those skilled in the art.

A heating system 156, for example, a multi-zone heater, can be controlled by the controller 120 via a data bus 152 to rapidly raise the temperature of the sample block 112 and/or the sample holders to higher incubation temperatures from lower incubation temperatures. The heating system 156 also may correct temperature errors in the upward direction during temperature tracking and control during incubations.

The heating system may include but is not limited to, for example, film heaters, resistive heaters, heated air, infrared heating, convective heating, inductive heating (e.g. coiled wire), Peltier based thermoelectric heating, and other heating mechanisms known to those skilled in the art. According to various exemplary embodiments, the cooling system and the heating system may be a single system configured to both increase and decrease the temperature of the block 112 and or of the sample holders directly.

In the exemplary embodiment of FIG. 1A, the controller 120 controls the temperature of the sample block 112 by sensing the temperature of the sample block 112 and/or fluid circulating within the sample block 112 via a temperature sensor 121 and the data bus 152 and by sensing the temperature of the cooling system 124 via bus 154 and a temperature sensor 161 in the cooling system 124. By way of example only, the temperature of the circulating fluid of the cooling system may be sensed, although other temperatures associated with the cooling system may also be sensed. In the exemplary embodiment of FIG. 1B, the controller 120 may control the temperature of the samples 110 by sensing the temperature of the samples 110 via a sensor 121 and the data bus 152. The sensor 121 in the embodiment of FIG. 1B may be, for example, a remote infrared temperature sensor or an optical sensor that detects a thermochromic dye in the samples 110. The controller 120 can also sense the internal ambient air temperature within the housing of the system via an ambient air temperature sensor 166. Further, the controller 120 can sense the line voltage for the input power on line 158 via a sensor 163. All these items of data together with items of data entered by the user to define the desired PCR protocol such as target temperatures and times for incubations are used by the controller 120 to carry out a desired temperature/time control program.

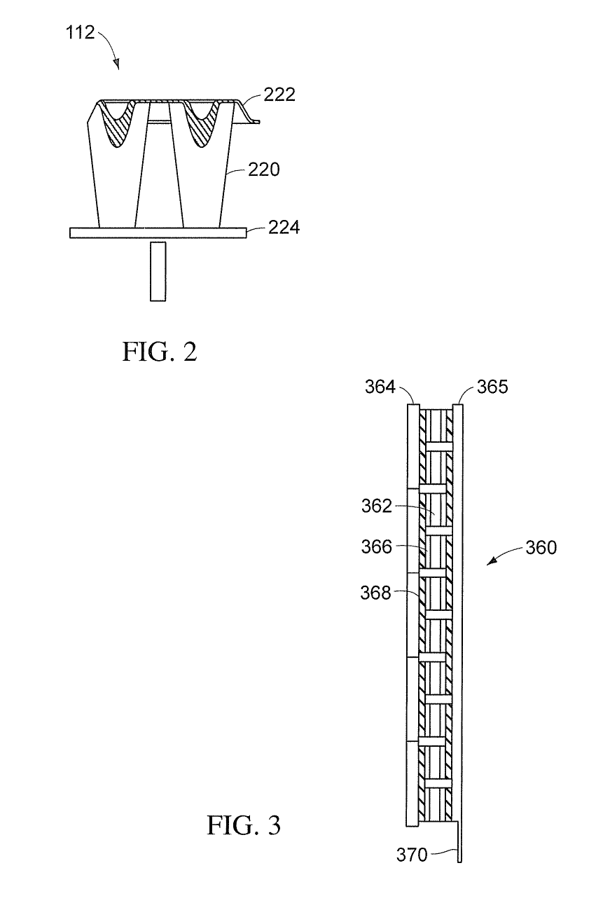

Referring now to FIG. 2, a cross-sectional view of a portion of an exemplary embodiment of the sample block 112 is illustrated. The sample block 112 can include a plurality of recesses 220 configured to accommodate the number and arrangement of the sample holder being used. For example, if a 96-well microtiter plate is being used, the sample block 112 may be provided with ninety-six (96) recesses 220 in a standard 12×8 configuration to accommodate, for example, the 96-well tray. Those having skill in the art would understand a variety of other configurations (e.g., number and arrangement) for the recesses 220 in order to accommodate other sample holder formats. Each of the recesses 220 may be configured to receive a sample well and/or capillary tube. The sample block 112 can include a one-piece structure including an upper support plate 222 and the recesses 220 may be fastened to a base plate 224, for example, by electroforming. The base plate 224 can provide lateral conduction to compensate for any differences in the thermal power output across the surface of each individual thermal electric device 360, shown in FIG. 3, and for differences from one thermal electric device to another. Alternatively, the sample block can be flat without recesses and configured to accommodate a microcard or flat-bottomed tray.

According to various exemplary embodiments, the heating system 156 may be, for example, a Peltier thermoelectric device 360, as shown in FIG. 3. The device 360 may include bismuth telluride couples 362 (for example, in the form of cube-like structures) sandwiched between two alumina layers 364, 365. The couples 362 can be electrically connected by solder joints 366 to copper traces 368 plated onto the alumina layers. One alumina layer can have an extension 370 to facilitate electrical connections. The thickness of the extended area can be reduced to decrease the thermal load of the device.

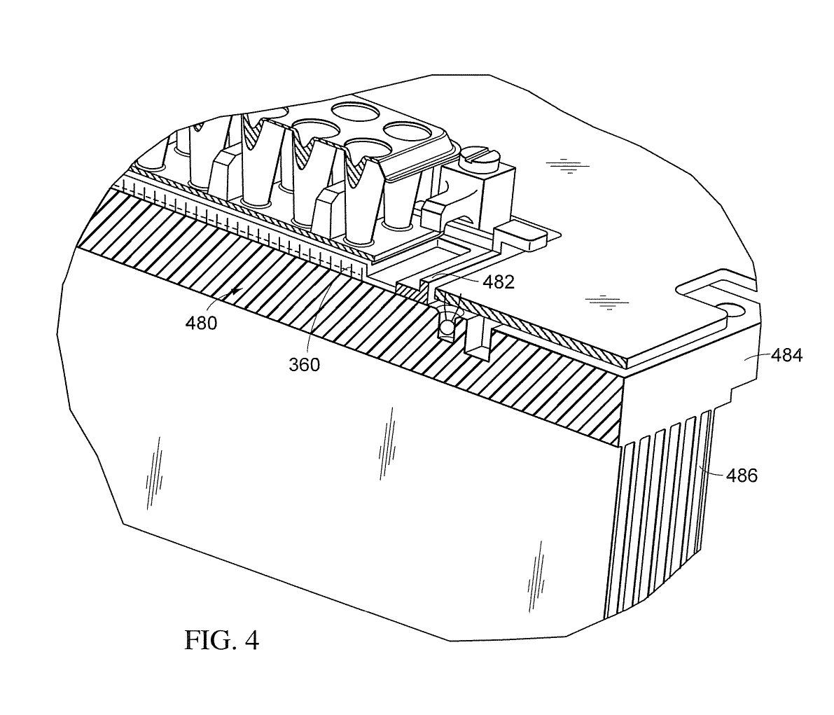

Referring now to FIG. 4, the cooling system 124 can comprise a heat sink 480 assembled with the thermoelectric device 360 and the sample block 112. A locating frame 482 can be positioned around the thermoelectric device 360 to align it with the sample block 112 and the heat sink 480 to maximize temperature uniformity across the sample block, when desired. The heat sink 480 can comprise a substantially planar base 484 and fins 486 extending from the base 484. The thermal mass of the heat sink is considerably larger than the thermal mass of the sample block 112 and samples 110 combined. As a result, the sample block 112 may change temperature significantly faster than the heat sink 480 for a given amount of heat transferred by the heating system 156.

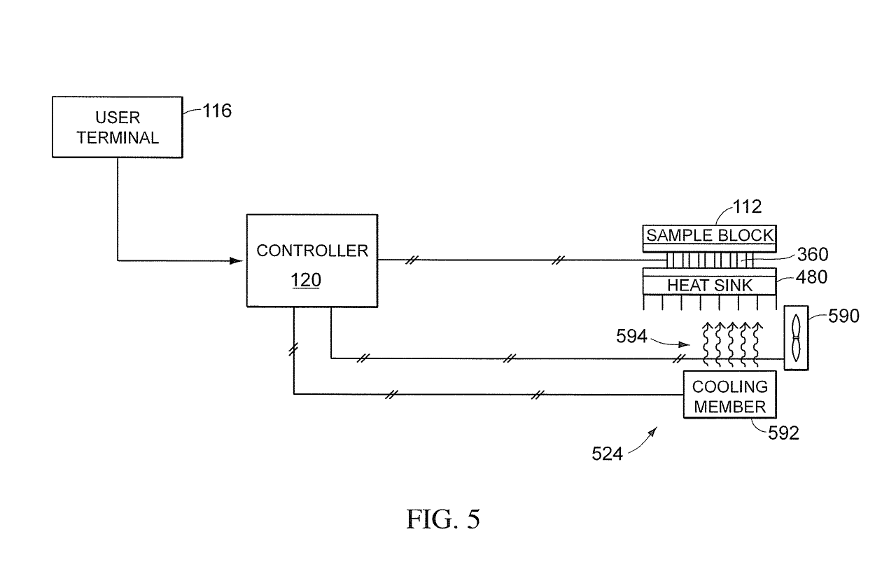

As shown in FIG. 5, according to some exemplary embodiments, a cooling system 524 can include a fan 590 and/or at least one cooling member 592 configured to control the heat sink temperature. The fan 590 and/or the cooling member 592 can be operably controlled, for example, by the controller 120. According to some aspects, the fan 590 and/or the cooling member 592 can be operated to hold the heat sink 480 at approximately 45° C., which is well within the normal PCR cycling temperature range. In some aspects, maintaining a stable heat sink temperature can improve repeatability of system performance.

According to some exemplary embodiments, the cooling member 592 can be configured to lower the temperature of the ambient air being directed toward the heat sink 480 by the conventional fan 590. As shown in FIG. 5, the cooling member 592 can lower the ambient air temperature by outputting a cooling fluid 594 such as, for example, CO₂ (bottled or dry), liquid nitrogen, pressurized air, a chilled gas (e.g., cold gas from liquid nitrogen), or the like into the airflow path of the fan 590.

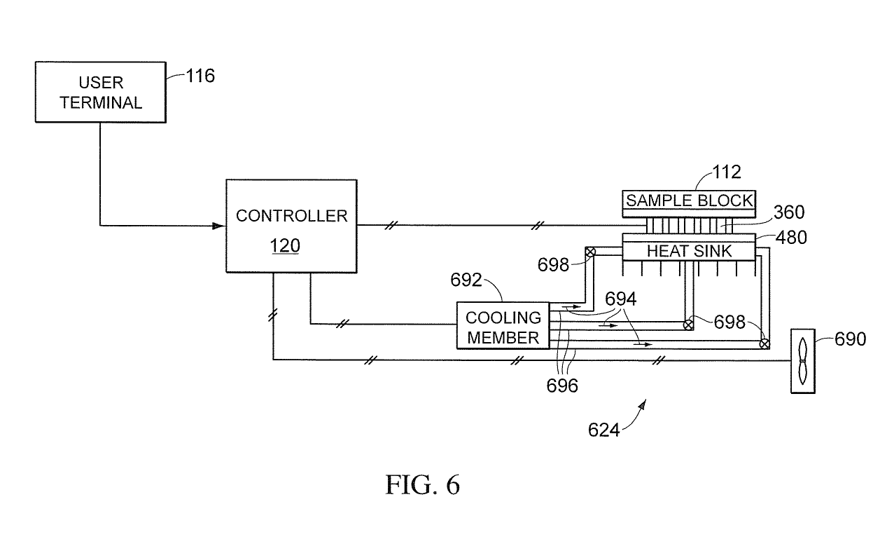

Referring now to FIG. 6, a cooling system 624 can comprise at least one cooling member 692 configured to output a cooling fluid 694 such as, for example, CO₂ (bottled or dry), liquid nitrogen, pressurized air, or the like to a series of plumbing 696 and valves 698 configured to direct the cooling fluid to one or more regions of the heat sink 480. According to some aspects, cooling system 624 can also include a conventional fan 690 to control the heat sink temperature.

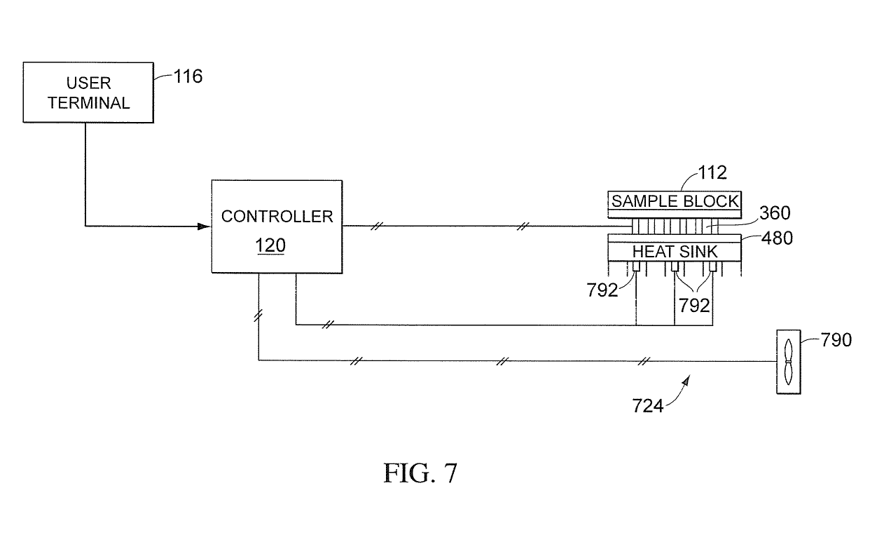

As shown in FIG. 7, according to various exemplary embodiments, a cooling system 724 can include one or more cooling members 792 configured to generate and/or direct cool air toward the heat sink 480 and/or to absorb heat from the heat sink 480. According to some aspects, one or more of the cooling members 792 can be mounted within the cooling fins 486 associated with a region of the sample block 112 so as to cool that specific region, as discussed below. According to some aspects, cooling system 724 can also include a conventional fan 790 to control the heat sink temperature.

Although the exemplary embodiments of FIGS. 5-7 show the use of a Peltier device 360 and heat sink 480, various other exemplary embodiments may include a cooling system comprising a cooling member that replaces the Peltier device and heat sink. Further, in systems wherein direct circulation of fluid around the sample holders is used for heating and/or cooling, a cooling system having a cooling member may be used in lieu of or in addition to such fluid circulation.

FIG. 10 depicts an exemplary embodiment of a cooling system 1024 comprising a cooling member 1092 and a conventional fan 1090. The cooling system 1024 may be configured to reduce the temperature of sample block 112 or of sample holder directly. The cooling member 1092 may thus be configured to output a cooling fluid such as, for example, CO₂ (bottled or dry), liquid nitrogen, pressurized air, or the like, in a manner similar to one or more of the cooling members 592, 692, 792. The cooling system 1024 also may be used in conjunction with a heating system (not shown in FIG. 10), such as, for example, the heating systems described herein, configured for raising the temperature of the block 112 or the sample holder directly. It will also be appreciated by those having skill in the art that, in accordance with various exemplary embodiments, the cooling systems 1024 may be used as the heating system as well, depending, for example, on the type of cooling member 1092 that may be used. Moreover, although the exemplary embodiments of FIGS. 5-7 and 10 illustrate a conventional fan 590, 690, 790, 1090 used in conjunction with the cooling systems 524, 624, 724, 1024, such a fan need not be utilized.

The term “cooling member” as used herein refers to cooling components that include devices other than Peltier devices, conventional fans, and/or conventional fluid circulation systems currently in use for reducing the temperature of samples during an incubation protocol in PCR thermal cycling devices and processes. Although the cooling systems discussed herein may use such a cooling member in combination with one or more of the above-listed conventional cooling mechanisms, a cooling member as used herein includes at least one component other than a conventional mechanism used for cooling in PCR thermal cycling. It is contemplated that cooling members used for cooling in PCR thermal cycling devices in accordance with exemplary embodiments of the invention may provide greater temperature control, improved efficiency, and/or improved heat transfer than the use of prior conventional cooling mechanisms.

According to various exemplary embodiments, the cooling member 592, 692, 792, 1092 may include, but is not limited to, one of several types of cooling components described in more detail below. As mentioned above, it is envisioned that the various cooling members described below may be used alone, in combination with conventional cooling mechanisms, such as, for example, conventional fans and/or Peltier devices, and/or in combination with one or more of the various other cooling members described below.

According to some exemplary aspects, the cooling member 592, 692, 792, 1092 can comprise one or more synthetic jet ejector arrays (SynJets), for example, as described in U.S. Pat. No. 6,588,497, which is incorporated herein by reference in its entirety. SynJets, developed at the Georgia Institute of Technology and licensed to Innovative Fluidics, are more efficient than conventional fans. For example, SynJets can produce two to three times as much cooling with two-thirds less energy input. The SynJets can comprise modules having a diaphragm mounted within a cavity having at least one orifice. Electromagnetic or piezoelectric drivers can cause the diaphragm to vibrate 100 to 200 times per second, rapidly cycling air into and out of the module and creating pulsating jets that can be directed to precise locations where cooling is needed. According to various aspects, the modules can be mounted directly within the cooling fins 486 of the heat sink 480.

According to various exemplary aspects, the cooling member 592, 692, 792, 1092 can comprise one or more vibration-induced droplet atomization (VIDA) devices, also developed at the Georgia Institute of Technology and licensed to Innovative Fluidics. VIDA devices use atomized liquid coolants, for example, water, to carry heat away from desired components. Piezoelectric actuators are used to produce high-frequency vibration to create sprays of tiny cooling fluid droplets inside a closed cell attached to an electronic component, for example, the heat sink 480, in need of cooling. The droplets form a thin film on the hot surface, for example, a hot surface associated with the heat sink 480, the metal block 112, or the sample holders, thereby allowing thermal energy to be removed by evaporation. The heated vapor then condenses, and the liquid is pumped back to the vibrating diaphragm for re-use. U.S. Pat. No. 6,247,525, incorporated herein by reference in its entirety, discloses exemplary embodiments of VIDA devices.

According to some exemplary aspects, the cooling member 592, 692, 792, 1092 can comprise a piezo fan. A piezo fan can be a solid state device comprising a compound piezo/stainless steel blade mounted to a PCB mount incorporating a filter and a bleed resistor. DC voltage can be delivered to an inverter drive circuit, which delivers a periodic signal to the fan that matches the resonant frequency of the fan, causing oscillating blade motion. The blade motion creates a high velocity flow stream from the leading edge of the blade that can be used to cool a heated surface, for example, the fins 486 of the heat sink 480, the metal block 112, or the surface of the sample holders. Piezo fans that may be utilized as the cooling member 592, 692, 792 can include, for example, those marketed by Piezo Systems, Inc.

According to various exemplary aspects, the cooling member 592, 692, 792, 1092 can comprise one or more Cold Gun Aircoolant Systems™, such as those marketed by EXAIR®. The Cold Gun uses a vortex tube, such as those marketed by EXAIR®, to convert a supply of compressed air into two low pressure streams—one hot and one cold. The cold air stream can be muffled and discharged through, for example, a flexible hose, which can direct the cold air stream to a point of use, for example, in the path of airflow from the fan 590, 690, 790, 1090 to a heated surface such as, for example, the fins 486 of the heat sink 480, the metal block, or the surface of the sample holders. Meanwhile, the hot air stream can be muffled and discharged via a hot air exhaust.

According to some exemplary aspects, the cooling member 592, 692, 792, 1092 can comprise one or more microchannel cooling loops, such as, for example, those marketed by Cooligy for use with high-heat semiconductors. An exemplary cooling loop can comprise a heat collector defined by fine channels, for example, 20 to 100 microns wide each, etched into a small piece of silicon, for example. In some embodiments, the channels can be configured to carry fluid that absorbs heat generated by a hot surface such as, for example, the heat sink 480, the metal block 112, or the sample holders. In some embodiments, the cooling loops can be configured to absorb heat from the ambient air in the path of airflow from the fan 590, 690, 790, 1090. The fluid passes a radiator, which transfers heat from the fluid to the air, thus cooling the fluid. The cooled fluid then return to a pump, for example, an electrokinetic pump, where it is pumped in a sealed loop back to the heat collector.

According to various exemplary aspects, the cooling member 592, 692, 792, 1092 can comprise one or more Cool Chips™, such as those marketed by Cool Chips plc. The Cool Chips™ use electrons to carry heat from one side of a vacuum diode to another. As such, Cool Chips™ are an active cooling technology, which can incorporate passive cooling components, such as the fan 590, 690, 790, 1090. A Cool Chip layer can be disposed between the heating system 156 and the heat sink 480 to introduce a gap between the heating system 156 and the heat sink 480 or between the heating system and the metal block 112 or sample holders. By addition of a voltage bias, electrons can be encouraged to move in a desired direction, for example, from the heating system 156 to the heat sink 480, while their return to the heating system 156 is deterred by the gap. Thus, the heat sink 480 can be hotter without damaging the heating system 156. In some aspects, one or more Cool Chips can be arranged to absorb heat from ambient air to thereby cool the system.

In some exemplary embodiments, carbon may be utilized to enhance temperature uniformity throughout the sample block 112. Since carbon transfers heat in two dimensions as opposed to three, it may be used to assist in heat transfer and in minimizing undesirable temperature variations throughout the sample block. By way of example, the heat sink 480, including, for example, fins 486, may comprise (e.g., be made from) carbon and/or carbon may be provided as an intermediate layer between the heat sink 480 and the cooling member 592, 692, 792, 1092 and/or carbon may be provided between the device 360 and the heat sink 480.

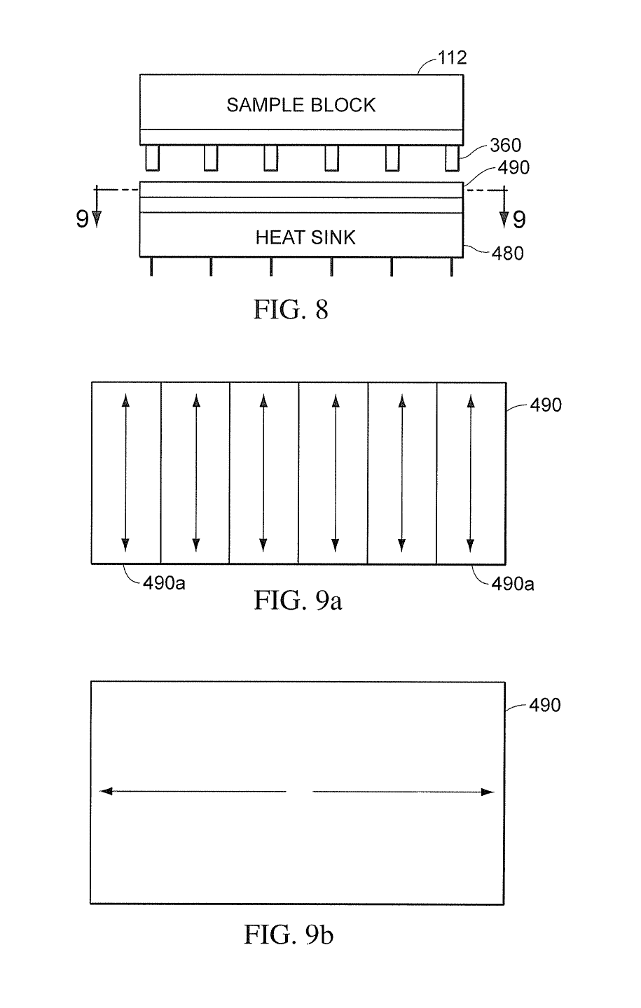

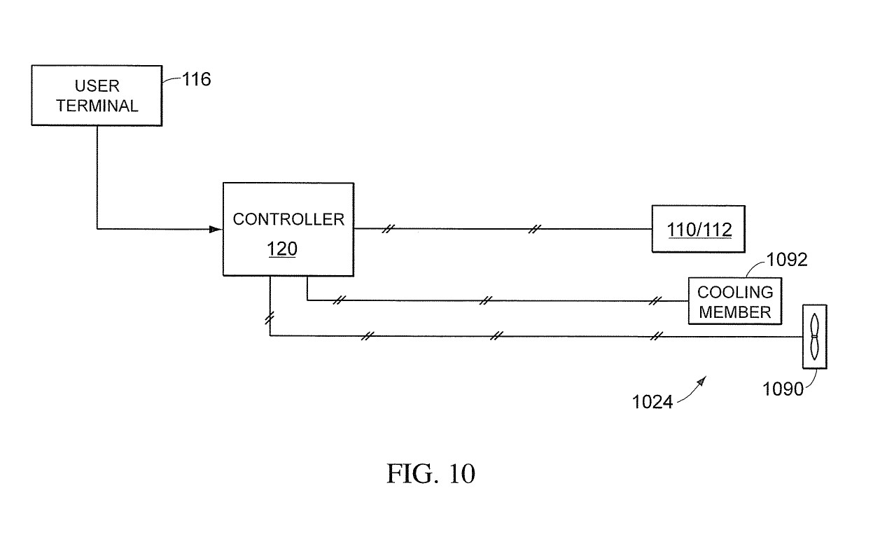

As depicted in FIG. 8, in some exemplary embodiments, the carbon may be substantially in the form of a block 490 provided as an intermediate layer between the heat sink 480 and device 360. The block 490 may be oriented so as to conduct heat in a vertical direction away from the sample block 112, although other orientations may be selected depending on the application and desired heat conduction. By way of example only, as shown in FIG. 9a, which is a view taken from line IX-IX in FIG. 8, the block 490 may comprise six 2×8 segments 490a forming a block 490 having overall 12×8 dimensions that correspond to the 12×8 sample block 112. Aside from conducting heat in a vertical direction (i.e., away from or toward the sample block 112 and heat sink 480), conduction in each segment 490a may take place along the long axis (i.e., in the direction of the arrows shown in FIG. 9a). In this manner, the end segments (e.g., the end segments 490a to the left and the right of the center of the block) would have a similar environment (e.g., temperature) as the center segments, which may minimize temperature variations between the center and end samples in the sample block 112. In another example, depicted in FIG. 9b, which also is view taken from line IX-IX in FIG. 8, the block 490 may be formed as a single piece and may be oriented so as to conduct heat in the vertical direction and along the long axis of the block 490, as depicted by the arrows in FIG. 9b. This orientation may minimize temperature variations across the sample block 112 (e.g., along a direction substantially perpendicular to the arrows shown in FIG. 9b.

Although the various cooling systems discussed above may reduce temperature nonuniformity experienced by the samples during temperature cycling of the samples through the various incubation stages, in some applications it may be desirable to induce controlled (e.g., predetermined) temperature gradients among the samples during the PCR protocol. It is envisioned that the various exemplary cooling members described above will assist in achieving desired temperature gradients due to the ability to exert greater control over the cooling effects produced by these cooling members. Thus, by controlling the cooling members through the controller and various bus lines and sensors, various regions the sample holders, the sample block 112, and/or the heat sink may be cooled by different amounts and/or rates in order to achieve desired temperature gradients among some or all of the samples 110.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “a biological” includes two or more different biological samples. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to the sample preparation device and method of the present disclosure without departing from the scope its teachings. Other embodiments of the disclosure will be apparent to those skilled in the art from consideration of the specification and practice of the teachings disclosed herein. It is intended that the specification and examples be considered as exemplary only.

1. A device for performing biological sample processing, the device comprising:
a sample holder configured to receive a biological sample;
a heating system configured to raise the temperature of the sample;
a cooling system configured to lower the temperature of the sample, wherein the cooling system comprises at least one cooling member; and
a controller configured to operably control the heating system and the cooling system to cycle the device through a desired time-temperature profile.

2. The device of claim 1, further comprising:
a sample block configured to be placed in thermal contact with the sample holder; and
a heat sink associated with the sample block,
wherein the heating system is configured to raise the temperature of at least a portion of the sample block, and
wherein the cooling system is configured to lower the temperature of at least a portion of the sample block and to lower the temperature of ambient air being directed toward the heat sink.

3. The device of claim 2, wherein the sample block comprises at least one recess configured to receive the sample holder.

4. The device of claim 2, further comprising a thermoelectric device positioned between the sample block and the heat sink.

5. The device of claim 1, wherein the at least one cooling member comprises a vibrating diaphragm system.

6. The device of claim 1, wherein the at least one cooling member comprises a vibration-induced droplet atomization system.

7. The device of claim 1, wherein the at least one cooling member comprises a piezo fan.

8. The device of claim 1, wherein the at least one cooling member comprises a micro channel liquid cooling system.

9. The device of claim 1, wherein the at least one cooling member is configured to direct one of carbon dioxide, liquid nitrogen, a chilled gas, and pressurized air into ambient air used for cooling the sample.

10. The device of claim 1, wherein the cooling system and the heating system comprise an integral system.

11. The device of claim 1, wherein the device is configured to perform polymerase chain reactions in a nucleic acid sample.

12. The device of claim 1, wherein the sample holder is configured to respectively support plural amounts of the biological sample at a plurality of locations of the sample holder.

13. A device for performing biological sample processing, the device comprising:
a sample holder configured to receive a biological sample;
a heating system configured to raise the temperature of the sample;
a cooling system configured to lower the temperature of the sample, wherein the cooling system comprises at least one cooling member selected from a synthetic jet ejector array, a vibration-induced droplet atomization system, a vibrating diaphragm system, a piezo fan, a Cold Gun, a microchannel cooling loop, and a Cool Chip; and
a controller configured to operably control the heating system and the cooling system to cycle the device through a desired time-temperature profile.

14. The device of claim 13, further comprising:
a sample block configured to be placed in thermal contact with the sample holder; and
a heat sink associated with the sample block,
wherein the heating system is configured to raise the temperature of at least a portion of the sample block, and
wherein the cooling system is configured to lower the temperature of at least a portion of the sample block and to lower the temperature of ambient air being directed toward the heat sink.

15. The device of claim 14, wherein the sample block comprises at least one recess configured to receive the sample holder.

16. The device of claim 14, further comprising a thermoelectric device positioned between the sample block and the heat sink.

17. The device of claim 14, wherein the at least one cooling member is configured to output a cooling fluid.

18. The device of claim 17, wherein the cooling fluid comprises one of carbon dioxide, liquid nitrogen, and compressed air.

19. The device of claim 17, further comprising a network of plumbing and valves configured to direct the cooling fluid from the at least one cooling member toward a region of the heat sink.

20. The device of claim 17, wherein the at least one cooling member is configured to direct the fluid toward a region of the heat sink.

21. The device of claim 14, wherein the heat sink comprises cooling fins, and the at least one cooling member is mounted within the cooling fins.

22. The device of claim 13, wherein the controller is configured to operably control the cooling system to achieve a predetermined temperature gradient among at least some of the samples.

23. The device of claim 13, wherein the device is configured to perform polymerase chain reactions.

24. The device of claim 13, wherein the sample holder is configured to respectively support plural amounts of the biological sample at a plurality of locations of the sample holder.

25. The device of claim 13, wherein the biological sample comprises a nucleic acid sample.

26. A device for performing biological sample processing, the device comprising:
means for holding a biological sample;
means for heating the sample;
means for cooling the sample, wherein the means for cooling the sample comprises a heat sink and a means for cooling the heat sink, wherein the means for cooling the heat sink comprises a cooling member; and
means for controlling the means for heating and the means for cooling to cycle the device through a desired time-temperature profile.

27. The device of claim 26, wherein the biological sample comprises a nucleic acid sample and wherein the device is configured to perform polymerase chain reactions.

28. A device for biological sample processing, the device comprising:
an enclosure configured to receive a biological sample for processing;
a heat sink in thermal communication with the enclosure; and
a thermal system configured to modulate a temperature of the at least one biological sample, the thermal system comprising a cooling system configured to lower a temperature of the at least one biological sample,
wherein the cooling system comprises a cooling fluid output configured to flow cooling fluid into a plurality of flow structures configured to respectively direct the cooling fluid to a plurality of differing locations of the heat sink, the cooling fluid being configured to absorb heat at the plurality of differing locations.

29. The device of claim 28, wherein the cooling fluid comprises at least one of air, water, a chilled gas, and carbon dioxide.

30. The device of claim 28, wherein the plurality of flow structures comprise flow channels.

31. The device of claim 28, further comprising a thermoelectric device in thermal communication with the enclosure.

32. The device of claim 28, wherein the enclosure is configured to receive a sample holder for holding plural amounts of the biological sample.

33. The device of claim 32, wherein the enclosure is configured to receive a sample holder comprising one of a microtiter plate, a microcard, a plurality of capillaries, and a plurality of tubes.

34. The device of claim 28, further comprising a sample holder configured to hold the biological sample for processing.

35. The device of claim 34, wherein the sample holder comprises one of a microtiter plate, a microcard, a plurality of capillaries, and a plurality of tubes.

36. The device of claim 28, further comprising a cover configured to form at least part of the enclosure.

37. The device of claim 28, further comprising a sample block configured to form at least part of the enclosure and to support the biological sample.

38. The device of claim 28, wherein the sample block is configured to transfer heat with the biological sample.

39. The device of claim 28, wherein the biological sample comprises a nucleic acid sample and wherein the device is configured to perform polymerase chain reactions on the nucleic acid sample.

40. The device of claim 28, further comprising a control system for controlling the thermal system.

41. The device of claim 28, wherein the thermal system further comprises a heating system configured to raise a temperature of the at least one biological sample.

42. The device of claim 284, wherein the cooling system and the heating system comprise an integral system.

43. The device of claim 28, wherein the heat sink comprises a plurality of projecting members configured to transfer heat away from the enclosure.

44. The device of claim 43, wherein the plurality of projecting members comprise a plurality of fins.

45. The device of claim 43, wherein the plurality of flow structures are configured to respectively direct the cooling fluid to differing groups of projecting members.

46. The device of claim 28, wherein the cooling system comprises at least one of a synthetic jet ejector array, a vibration-induced droplet atomization system, a vibrating diaphragm system, a piezo fan, a Cold Gun, a microchannel cooling loop, and a Cool Chip.

47. The device of claim 28, wherein the cooling system comprises a fan.

48. A method for performing biological sample processing, the method comprising:
supplying an enclosure with a biological sample for processing within the enclosure;
modulating a temperature of the biological sample to cycle a temperature of the biological sample,
wherein modulating the temperature of the biological sample comprises respectively directing a cooling fluid via a plurality of separate flow passages to a plurality of locations of a heat sink in thermal communication with the enclosure, wherein the plurality of locations are independently cooled via the cooling fluid respectively directed to each of the plurality of locations.

49. The method of claim 48, wherein supplying the enclosure comprises supplying a sample holder holding the biological sample to the enclosure.

50. The method of claim 49, wherein supplying the sample holder comprises supplying one of a microtiterplate, a microcard, a plurality of capillaries, and a plurality of tubes holding the biological sample.

51. The method of claim 48, wherein modulating the temperature of the biological sample comprises modulating the temperature via a thermoelectric device.

52. The method of claim 48, wherein modulating the temperature of the biological sample further comprises heating the biological sample.

53. The method of claim 48, wherein modulating the temperature of the biological sample further comprises controlling a temperature of a sample block in thermal communication with the biological sample.

54. The method of claim 48, wherein directing the cooling fluid comprises directing a cooling fluid comprising one of air, water, a chilled gas, and carbon dioxide.

55. The method of claim 48, further comprising performing polymerase chain reactions on the biological sample supplied to the enclosure.

56. The method of claim 48, wherein supplying the enclosure with the biological sample for processing within the enclosure comprises supplying the enclosure with a nucleic acid sample.

57. The method of claim 48, wherein directing the cooling fluid comprises directing a cooling fluid output from one of a synthetic jet ejector array, a vibration-induced droplet atomization system, a vibrating diaphragm system, a piezo fan, a Cold Gun, a microchannel cooling loop, and a Cool Chip.

58. The method of claim 48, wherein modulating the temperature of the biological sample comprises circulating air from a fan to provide cooling.
